(19) United States
(12) Patent Application Publication
(10) Pub. No.: US 2021/0110923 A1
(43) Pub. Date: Apr. 15, 2021

(54) SHARED DISTRIBUTED EMERGENCY SYSTEM AND METHOD OF MANAGING EMERGENCY EVENTS

(71) Applicant: Unify Patente GmbH & Co. KG, Munchen (DE)

(72) Inventor: Ioannis Kolaxis, Athens (GR)

(21) Appl. No.: 16/653,215

(22) Filed: Oct. 15, 2019

(51) Int. Cl. G16H 40/67; H04L 29/08; G06Q 20/14

(52) U.S. Cl. CPC G16H 40/67; G06Q 20/14; H04L 67/10

(57) ABSTRACT

An emergency system can include a server adapted to receive information on emergency events via a communication network. There can be a database populated with data received via the first communication network relating to the emergency events that is adapted to provide different access rights to various entities that are interested in the data relating to the emergency events stored in the database. The database can be structured to implement private blockchain technology to prevent modification of the stored data. A method of managing emergency events using an embodiment of the shared distributed emergency system can include use of the database. The server can be configured to host the database or facilitate other computer devices' access to the database for reading access or for reading/writing access to the data of the database.

### FIELD

The present invention relates to a shared distributed emergency system, a communication apparatus configured to facilitate a shared distribution of emergency related information, and to a method of managing emergency events using a shared distributed emergency system.

### BACKGROUND

Every day, a large number of emergencies such as car accidents or other accidents happen that entail substantial administrative work for a number of parties involved. For example, regarding a car crash, 9-1-1 will receive a phone call and depending on the severity of the car crash, 9-1-1 may have to send out the police, an ambulance and the fire brigade. Each one of them (police, ambulance, fire brigade) will have to file a separate report for the very same emergency event, mostly repeating information related to the event, such as the names of the persons involved, the license plates of the cars involved in the car crash, etc.

Thereby, every authority involved in handling the emergency event, as police, ambulance, and fire brigade, basically has to do the same administrative work, as filing a report for this emergency event, and listing the personal data of people involved. The same applies for the insurance companies involved in such emergency events, as for example, the car insurance as well as health/life insurance, in case of injuries or casualties so as to process the resulting claims.

Moreover, a separate report will have to be filed for each car and individual that has been involved in the car crash. It was determined that if the time that is needed by every single person (police, ambulance, fire brigade, insurance companies) involved in the administrative process of handling an emergency, then this duplicative work accumulates, resulting in high administrative costs.

Additionally, it is likely that mistakes or inconsistencies may occur in the data that are being manually replicated in different reports for the same event. For example, a license plate or a name may be mistaken in one of the reports. Even worse, a report may be falsified by one of the involved parties, e.g., for fraudulent insurance claims. It was determined that a new apparatus and method are needed to better account for these problems that were identified.

### SUMMARY

According to one aspect of the present invention, a shared distributed emergency system is provided, comprising a at least one server, which is adapted to receive information on emergency events via a communication network, wherein the emergency system comprises a distributed database, the database being
populated with data received via the first communication network relating to the emergency events,
adapted to provide different access rights to various entities that are interested in the data relating to the emergency events stored in the database, and
to implement private blockchain technology so as to prevent modification of the stored data.

Thus, according to this aspect of the present invention, an emergency system is enhanced by providing a distributed database, where all the emergency events are being registered by the authorities. Each party (police, ambulance, fire brigade, insurance companies) will have access to this distributed database with different access rights. For example, each one of them may be able to read common data (such as the license plates of the cars involved in an accident, or the names of the persons driving the corresponding cars), but only medical professionals (from the ambulance) & health insurance employees will be able to read personal data related to the health status (e.g. injuries due to the car accident) of an individual.

The inventive emergency system comprising such a database will save valuable administrative time from every individual that is involved in handling emergencies, thus allowing them to use this time in other activities. As an example, less officers would be needed to do paper work, freeing up officers to fight crime. Since the database is immutable due to blockchain technology, nobody will be able to modify or delete existing data, therefore eliminating fraudulent behaviour.

According to a preferred embodiment of the invention, the database is a distributed database. Preferably, the emergency system is OpenScape First Response.

It should be appreciated that embodiments of the emergency system can include communication devices. For instance, the database can be a distributed database hosted by a database server (e.g. a computer device having non-transitory memory, a processor, and at least one transceiver having at least one receiver and at least one transmitter). The database hosting computer device can be communicatively connectable to computer devices of different entities (e.g. a computer device of a police department, a computer device of a hospital system, a computer device of a fire brigade system, a computer device of an insurance provider, etc.). Each of these computer devices can be communicatively connectable via at least one network connection, for instance, There may be one or more border control devices, firewalls, access points, routers, and/or gateways included in the systems via which each computer device is connected to the computer device hosting the distributed database.

According to a further preferred embodiment of the invention, the blockchain is applied to the emergency system, in particular, to OpenScape First Response. Therefore, by enhancing currently implemented emergency systems such as OpenScape First Response by bundling them with a blockchain that will be accessible by all the interested organizations, every organization will be able to achieve higher productivity, innovation and success.

According to a still further preferred embodiment of the invention, the emergency information is provided by public facilities or by emergency responders or by private users.

Preferably, the access right to the database is charged with a fee corresponding to the type of access right. Namely, the database will be basically populated with data coming from the emergency responders. However, since insurance companies will also be using it to make their job more effective, they will have to pay a fee for the maintenance of this distributed database. Essentially everybody wins: the insurance companies that will work more effectively, eliminating the possibility of fraudulent claims, and the emergency authorities (police, ambulance, etc.) that will have to pay less for the maintenance of those systems, since the insurance companies will also contribute. This will allow for huge savings in the administrative process of handling emergency events and will lower the “Total Cost of Ownership” (TCO) of the emergency systems for the state authorities.

Also, the type of access rights may comprise a first type of access right allowing to write data into the database and to read data from the database, and a second type of access right allowing to only read data from the database. Further, the database may comprise a first section comprising common data and a second section comprising personal data, wherein the common data comprises data relating to license plates of cars involved in an accident, and/or names and/or addresses of persons involved in the accident.

It is advantageous, if the personal data comprises data relating to the health status of persons involved in the accident.

Moreover, the access rights may be linked to the first section and the second section, respectively, of the database.

According to a further aspect of the present invention, a method of managing emergency events using a shared distributed emergency system is provided, wherein the method comprises the steps of:
receiving information on emergency events via a first communication network, and which is adapted to communicate with a communication platform, in which a number of clients communicates with a server via a second communication network,
populating a distributed database with the information on emergency events received,
providing different access rights to the various entities that are interested in the data relating to the emergency events stored in the database, and
employing a private blockchain as a distributed database so as to prevent modification of the stored data.

Preferably, the method may further comprise a step of assigning the data received to a first section of the database comprising common data and a second section of the database comprising personal data.

Moreover, the method may comprise a step of charging a fee depending on the type of access right provided to an entity that is interested in the data relating to the emergency events.

Other details, objects, and advantages of the telecommunications apparatus method will become apparent as the following description of certain exemplary embodiments thereof proceeds.

### BRIEF DESCRIPTION OF THE DRAWINGS

The invention and embodiments thereof will be described below in further detail in connection with the drawing.

FIG. 1 schematically illustrates an embodiment of a shared distributed emergency system according to an embodiment of the invention.

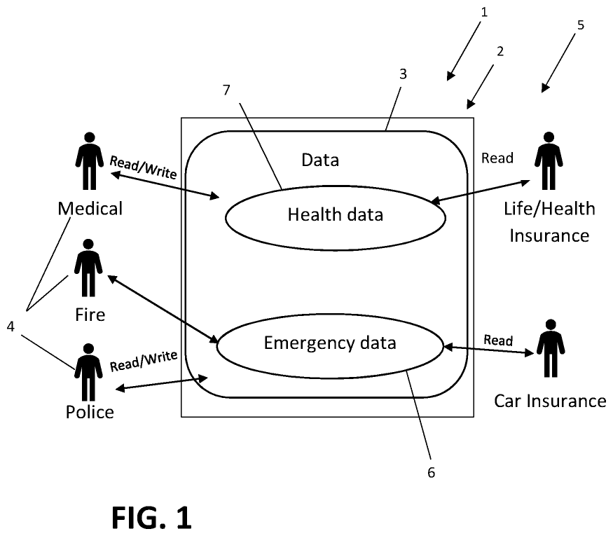

Reference numerals utilized in FIG. 1 include:
1 emergency system;
2 server;
3 database;
4 emergency responder;
5 communication network;
6 first section of database; and
7 second section of database;

### DETAILED DESCRIPTION OF EXEMPLARY EMBODIMENTS

FIG. 1 schematically illustrates a shared distributed emergency system 1 according to an embodiment of the invention. The shared distributed emergency system 1, which may be realized for example by OpenScape First Response, comprises a server 2 with a database 3, in which information on emergency events is stored, as will be outlined in further detail below. The server 2 can include at least one computer device that includes at least one processor connected to non-transitory memory and at least one transceiver unit. The transceiver unit can include at least one receiver and at least one transmitter to facilitate communications via at least one type of network connection with other computer devices (e.g. terminal devices, smart phones, personal computers, laptops, etc.). The server 2 can be accessible via the internet, for example, and be connectable to other computer devices via one or more gateways and/or other nodes that can facilitate communications with the server via the internet from those computer devices' enterprise networks (e.g. session border control devices, routers, firewall devices, etc.).

The server 2 can be configured to receive data relating to emergency events, for example, from emergency responders 4 via a communication network 5. The communication network 5 can be configured as a wide area network (WAN), or other type of network (e.g. local area network, large area network, enterprise network, a collection of inter-connected enterprise communication networks, etc.).

The communication network 5 can be configured so that other entities, authorities, or private persons may provide information on emergency events to be input into the database 3 via their respective computer devices. It is noted that in order to prevent modification of the data stored in the database so as to prevent fraud, a private blockchain can be employed in place of the database 3.

As can be seen from FIG. 1, the database 3 which is a distributed database, comprises a first section 6 comprising common emergency data, as for example, data relating to license plates of cars involved in an accident, and/or names and/or addresses of persons involved in the accident, and a second section 7 comprising personal health data, as for example, data relating to the health status of persons involved in the accident.

Also, the emergency system 1 according to an embodiment of the invention is adapted to provide different access rights to the various entities or persons accessing the database 3, i. e. to entities or persons that are interested in the data relating to emergency events stored in the database 3. According to an embodiment of the invention, the access rights may be subdivided into a first type of access right allowing to write data into the database as well as to read data from the database, and a second type of access right allowing to only read data from the database. For example, first responders or police agents or the fire department may be not only be allowed to read data but also be allowed to write data relating to an accident into the database 3, for example, into the first section 6 comprising the common emergency data, whereas medical staff may be allowed to read data and write data relating to an accident into the database 3, for example, into the second section 7 comprising the personal health data. Further, insurance companies may only be provided with an access right allowing to read data from the second section 7, whereas car insurance companies are only provided with an access right allowing to read data from the first section 6 of the database 3. Also, corresponding to the respective access right, the authorities or entities using the database 3 as outlined above, may be charged with different fees. For example, the insurance companies may be charged with higher fees than the emergency authorities, as police, ambulance, or fire brigade.

Summarizing the above, an embodiment of an emergency system can be provided to which the emergency authorities as well as the insurance companies will have access to a shared, distributed database, which enables them to achieve huge savings in the administrative process of handling emergency events, effectively lowering the “Total Cost of Ownership” (TCO) of the emergency systems for the state authorities. Data on emergency events is collected and stored and may be accessed, as needed. Thus, redundant administrative work for collecting data on accidents or other emergencies can be avoided. Also, since blockchain technology can be used in combination with the distributed database, the stored data can be immutable, i.e., existing data cannot be modified and can serve as the single source of truth for all the stakeholders (emergency authorities, insurance companies, etc.) who need to process information regarding emergency events.

It is contemplated that a particular feature described, either individually or as part of an embodiment, can be combined with other individually described features, or parts of other embodiments. The elements and acts of the various embodiments described herein can therefore be combined to provide further embodiments. Thus, while certain exemplary embodiments of a shared distributed emergency system, a telecommunications apparatus and methods of making and using the same have been shown and described above, it is to be distinctly understood that the invention is not limited thereto but may be otherwise variously embodied and practiced within the scope of the following claims.

What is claimed is:

1. A shared distributed emergency system comprising a server, which is adapted to receive information on emergency events via a communication network, the emergency system comprising a distributed database, the database being
populated with data received via the first communication network relating to the emergency events,
adapted to provide different access rights to various entities that are interested in the data relating to the emergency events stored in the database, and
to implement blockchain technology so as to prevent modification of the stored data.

2. The shared distributed emergency system according to claim 1, wherein the database is a distributed database.

3. The shared distributed emergency system according to claim 1, wherein the emergency system is OpenScape First Response.

4. The shared distributed emergency system according to claim 3, wherein the blockchain is deployed to the emergency system, in particular, to OpenScape First Response.

5. The shared distributed emergency system according to claim 1, wherein the emergency information is provided by public facilities or by emergency responders or by private users.

6. The shared distributed emergency system according to claim 1, wherein the access right to the database is charged with a fee corresponding to the type of access right.

7. The shared distributed emergency system according to claim 1, wherein the type of access rights comprises a first type of access right allowing to write data into the database and to read data from the database, and a second type of access right allowing to only read data from the database.

8. The shared distributed emergency system according to claim 1, wherein the database comprises a first section comprising common data and a second section comprising personal data.

9. The shared distributed emergency system according to claim 8, wherein the common data comprises data relating to license plates of cars involved in an accident, and/or names and/or addresses of persons involved in the accident.

10. The shared distributed emergency system according to claim 8, wherein the personal data comprises data relating to the health status of persons involved in the accident.

11. The shared distributed emergency system according to claim 8, wherein the access rights are linked to the first section and the second section, respectively, of the database.

12. A method of managing emergency events using a shared distributed emergency, wherein the method comprises the steps of
receiving information on emergency events via a first communication network, and which is adapted to communicate with a communication platform, in which a number of clients communicates with a server via a second communication network,
populating a distributed database with the information on emergency events received,
providing different access rights to the various entities that are interested in the data relating to the emergency events stored in the database, and
using a private blockchain as the database so as to prevent modification of the stored data.

13. The method according to claim 12, wherein the method further comprises a step of assigning the data received to a first section of the database comprising common data and a second section of the database comprising personal data.

14. The method according to claim 12, wherein the method further comprises a step of charging a fee depending on the type of access right provided to an entity that is interested in the data relating to the emergency events.
